(19) United States
(12) Patent Application Publication
(10) Pub. No.: US 2002/0010125 A1
(43) Pub. Date: Jan. 24, 2002

(54) Assay for agents that induce chemokinesis

(76) Inventors: Dennis A. Carson, Del Mar, CA (US); Lorenzo M. Leoni, San Diego, CA (US); Howard B. Cottam, Escondido, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/810,010

(22) Filed: Mar. 16, 2001

Related U.S. Application Data
(60) Provisional application No. 60/189,976, filed on Mar. 16, 2000.

(51) Int. Cl. A61K 31/00; C12Q 1/00; C12N 5/08

(52) U.S. Cl. 514/1; 435/4; 435/372

(57) ABSTRACT

The present invention provides methods for identifying compounds that can induce cellular chemokinesis. According to the present invention, chemokinesis interferes with immune and inflammatory responses by increasing cell movements and altering cell migration patterns. Surprisingly, compounds isolated according to the present invention can interfere with the spread of malignant cells through the body, reduce inflammatory responses and can cause leukocytes to be retained in lymph nodes, the spleen and other organs of the reticulo-endothelial system. Several methods are contemplated by the present invention for identifying compounds which can induce chemokinesis. In one embodiment the method involves contacting a population of target cells with a test compound and observing whether the target cells produce a chemotactic molecule; wherein the target cell has a cognate receptor for the chemotactic molecule. In another embodiment, the method involves contacting a population of target cells with a test compound and observing whether the target cells homotypically aggregate. In yet another embodiment, the method involves contacting a population of target cells with a test compound and observing whether actin filaments in the target cells form stress fibers.

### RELATED APPLICATIONS

This Application claims the benefit of U.S. Provisional Application No. 60/189,976 filed Mar. 16, 2000 under 35 U.S.C. 119(e).

### FIELD OF THE INVENTION

The present invention provides methods for identifying compounds that can induce cellular chemokinesis. According to the present invention, chemokinesis interferes with deleterious immune and inflammatory responses by increasing cell movements and altering cell migration patterns. Compounds isolated according to the present invention can interfere with the spread of malignant cells through the body, reduce inflammatory responses and can cause leukocytes to be retained in lymph nodes, the spleen and other organs of the reticulo-endothelial system. Such compounds are useful for the treatment of inflammatory, allergic, autoimmune, and degenerative diseases. The present compounds are also useful for the prevention of allograft rejection, for wound repair, for recovery from injury, and for the therapy of lymphoproliferative diseases and cancer.

### BACKGROUND OF THE INVENTION

Chemotaxis is broadly defined as the directed orientation or movement of an organism or cell in relation to a chemical agent. Cellular chemotaxis assay procedures are available and, for example, can test whether a cell will move from a chamber containing no chemoattractant to another chamber which contains a chemoattractant. Chemokinesis, on the other hand, is the undirected movement of a cell or organism in response to a chemokine. It is distinct from chemotaxis in that the chemokinetic movement is random; the cell or organism does not move toward a chemoattractant or away from a chemorepellant. The cell or organism merely begins to move more than it would otherwise.

Leukocyte chemotaxis is believed to be necessary for development of inflammatory and other immune responses. Leukocyte cell classes that participate in cellular immune responses include lymphocytes, monocytes, neutrophils, eosinophils and mast cells. Lymphocytes are “master cells” that control the activity of most of these other cell types, particularly the monocytes. Lymphocytes are the leukocyte class that initiate, coordinate, and maintain the inflammatory response. Lymphocytes attract monocytes to the site that cause much of the actual tissue damage occurring during inflammatory diseases. Leukocytes accumulate at a site of inflammation release granular contents, various hydrolytic enzymes and other toxic components into the extracellular spaces. As a result, the surrounding tissue is damaged. Numerous chronic inflammatory disease are thought to involve the aberrant presence leukocytes in tissues. Rheumatoid arthritis, osteoarthritis and psoriasis are a few examples. The lung is particularly vulnerable. Aggregation and migration of leukocytes into this organ can lead to microvascular occlusion, endothelial damage and subsequent edema. Infiltration of these cells is responsible for a wide range of chronic inflammatory and autoimmune diseases, and also organ transplant rejection. These diseases include rheumatoid arthritis, psoriasis, contact dermatitis, inflammatory bowel disease, multiple sclerosis, atherosclerosis, sarcoidosis, idiopathic pulmonary fibrosis, dermatomyositis, hepatitis, diabetes, allograft rejection, and graft-versus-host disease.

The process by which leukocytes leave the bloodstream and accumulate at inflammatory sites, and initiate disease, is mediated at a molecular level by chemoattractant receptors, by cell-surface proteins called adhesion molecules, and by the ligands that bind to these two classes of cell-surface receptor. Over the last few years, many chemotactic ligands, called chemokines, have been identified which can bind to chemoattractant receptors and thereby induce chemotaxis. Chemokines are secreted in response to an insult to the immune system by proinflammatory cells, leukocytes or endothelial cells. Chemokine proteins are classified into four families based upon the location of the first conserved cysteine residue in the protein. These families of chemokine proteins are described in more detail in Ziotnik et al., 12 Immunity 121-27 (2000) and Saunders et al., 4 DDT 80-92 (1999).

Chemokines are generally perceived to have a profound influence over the selective recruitment of specific cell types during an acute inflammatory disease and methods have been developed to inhibit chemokine activity in order to treat such diseases. To disturb chemotaxis, investigators have focused previously on agents that (a) neutralize chemokines or other cell attractive molecules, or (b) prevent the stable adherence of leukocytes (or malignant cells) to endothelial cell integrins and selecting. While some of these approaches have achieved limited success, these approaches suffer from the problem that many distinct molecules have chemotactic activity and treatment approaches that involve only one or a few of these molecules have little effect. For example, more than thirty protein chemokines, several different inflammatory mediators, and various low molecular weight compounds have been identified. Neutralization of only one chemotactic substance, therefore, does not totally disrupt directed cell migration. Accordingly, new approaches are needed that circumvent the necessity to neutralize every type of cytokine in order to effectively treat chronic inflammatory and related diseases.

### SUMMARY OF THE INVENTION

According to the present invention, compounds which can induce chemokinesis increase cell movements and alter cell migration patterns, thereby interfering with harmful immune and inflammatory responses. Surprisingly, compounds isolated according to the present invention can interfere with the spread of malignant cells through the body, reduce inflammatory responses and can cause leukocytes to be retained in lymph nodes, the spleen and other organs of the reticulo-endothelial system. Such compounds are useful for the treatment of inflammatory, allergic, and autoimmune diseases, for the prevention of allograft rejection, and for the therapy of lymphoproliferative diseases and cancer.

The present invention provides a method of identifying compounds that induce chemokinesis which includes observing whether a test compound promotes random movement of a target cell. The present invention is also directed to compounds isolated by these methods.

The present invention also provides a method of identifying compounds that induce chemokinesis which includes observing whether a test compound stimulates a target cell to produce chemotactic molecules; wherein the target cell has a cognate receptor for the chemotactic molecule. The present invention is also directed to compounds isolated by these methods.

The present invention further provides a method of identifying compounds that induce chemokinesis which includes observing whether a test compound causes homotypic aggregation of target cells; wherein such homotypic aggregation by the target cells is blocked by a secretion inhibitor. Examples of secretion inhibitors include brefeldin A and monensin. The present invention is also directed to compounds isolated by these methods.

The present invention still further provides a method to determine the ability of a test compound to induce chemokinesis in a population of leukocytes which includes:

(a) contacting a population of cells with an amount of a test compound in vitro; and

(b) determining the ability of the test compound to induce a chemokinetic response in the cells, wherein the response is indicative of the ability of the test compound to reduce the level of circulating leukocytes in vivo.

An increased chemokinetic response is indicated, for example, when an increased number of cells pass from a chamber containing the cells in an appropriate medium through a microporous membrane. Thus, a chemokinetic response occurs when the number of cells passing through the microporous member following step (a) above, is greater than the number passing through said membrane prior to step (a). An increased chemokinetic response is also indicated, for example, when cellular migration through a microporous membrane is enhanced by a chemotaxin on the side of the membrane opposite a chamber containing the cells.

The present invention also provides a therapeutic method for treating a condition ameliorated by induction of chemokinesis in a specific cell type of a mammal which includes systemically administering to a mammal afflicted with said condition a pharmaceutically effective amount of a compound identified by the methods of the present invention. The specific cell type can be, for example, lymphocyte, neutrophil, basophil, eosinophil, monotcyte, CD4 T cell lymphocyte, CD8 T cell lymphocyte or B lymphocyte. The compound can be etodolac.

The present invention further provides a method of stimulating chemokinesis in a specific cell type of a mammal which includes systemically administering a pharmaceutically effective amount of a compound identified by the methods of the present invention. The specific cell type can be, for example, lymphocyte, neutrophil, basophil, eosinophil, monotcyte, CD4 T cell lymphocyte, CD8 T cell lymphocyte or B lymphocyte. The compound can be etodolac.

The present invention also provides a method of treating inflammation in a specific cell type of a mammal which includes stimulating chemokinesis in that cell type by systemically administering a pharmaceutically effective amount of a compound identified by the methods of the present invention. The specific cell type can be, for example, lymphocyte, neutrophil, basophil, eosinophil, monotcyte, CD4 T cell lymphocyte, CD8 T cell lymphocyte or B lymphocyte.

The present invention also provides a method of inhibiting malignant cancer cell metastatis in a mammal which includes stimulating chemokinesis in an identified malignant cancer cell type by systemically administering a amount of a compound identified by the methods of the present invention which is effective to induce chemokinesis in the malignant cancer cell.

The present invention further provides a method of depleting chronic lymphocytic leukemia cells in a mammal which includes stimulating chemokinesis in chronic lymphocytic leukemia cells of a mammal by systemically administering to the mammal an amount of a compound identified by the methods of the present invention which is effective to induce chemokinesis in leukocytes associated with leukemia.

The present invention also provides a method of inducing cytoskeletal changes in colon cancer cells of a mammal which includes stimulating chemokinesis in a colon cancer cell type by systemically administering an amount of a compound identified by the methods of the present invention which is effective to induce chemokinesis in the colon cells.

The present invention also provides a method of treating transplant rejection in a mammal which includes stimulating chemokinesis in lymphocytes of said mammal by systemically administering to said mammal a pharmaceutically effective amount of a compound identified by the methods of the present invention. In one embodiment the lymphocyte is CD8 T cell lymphocyte and the compound is etodolac.

The present invention also provides a method of treating an allergy in a mammal which includes stimulating chemokinesis in leukocytes of the mammal by systemically administering to the mammal a pharmaceutically effective amount of a compound identified by the method of the present invention. The leukocyte can, for example, be basophil, eosinophil or CD4 T cell lymphocyte and the compound can be etodolac.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### DESCRIPTION OF THE DRAWINGS

FIG. 1 is a bar graph illustrating that the number of chronic lymphocytic leukemia cells migrating through a 5 to 8 micron membrane increases when the cells are cultured in the presence of a test compound (etodolac). Chronic lymphocytic leukemia cells were incubated overnight in medium containing the indicated enantiomer of etodolac at either 0.0 μmol (blue bar) or 200 μmol (green bar) concentrations. The cells were then added to the upper wells of chemotaxis chambers divided by a 5 to 8 micron membrane. No etodolac was present in the lower portion of each well. The bar graph provides the numbers of cells crossing through the membranes into the lower well.

FIG. 2 is a bar graph illustrating that the number of peripheral blood lymphocytes migrating through a 5 to 8 micron membrane increases when the cells are cultured in the presence of a test compound (etodolac). Peripheral blood lymphocytes were incubated overnight in medium containing the indicated enantiomer of etodolac at either 0.0 μmol (blue bar) or 250 μmol (green bar) concentrations. The cells were then added to the upper wells of chemotaxis chambers divided by a 5 to 8 micron membrane. No etodolac was present in the lower portion of each well. The bar graph provides the numbers of cells crossing through the membranes into the lower well.

FIG. 3 is a bar graph illustrating that the number of chronic lymphocytic leukemia cells migrating through a 5 to 8 micron membrane increases when the cells are cultured and tested in the presence of a test compound (etodolac). Chronic lymphocytic leukemia cells were incubated overnight in medium containing the indicated enantiomer of etodolac at 12.5 μmol (blue bar), 25 μmol (green bar), 50 μmol (bright red bar), 100 μmol (yellow bar), 250 μmol (dark red bar) or 500 μmol (orange bar) concentrations. The cells were then added to the upper wells of chemotaxis chambers divided by a 5 to 8 micron membrane. The lower portion of each well contained interferon-inducible protein of 10 kd (IP-10) which is a chemokine associated with inflammatory disease. The IP-10 chemokine was used because chronic lymphocytic leukemia cells express high levels of the CXCR3 chemokine receptor and are attracted by IP-10. The bar graph provides the numbers of cells crossing through the membranes into the lower well.

FIG. 4 is a bar graph illustrating that the number of peripheral blood lymphocytes migrating through a 5 to 8 micron membrane increases when the cells are cultured and tested in the presence of a test compound (etodolac). Peripheral blood lymphocytes were incubated overnight in medium containing the indicated enantiomer of etodolac at 0.0 μmol (blue bar), 50 μmol (green bar), 100 μmol (bright red bar), 250 μmol (yellow bar) or 500 μmol (dark red bar) concentrations. The cells were then added to the upper wells of chemotaxis chambers divided by a 5 to 8 micron membrane. The lower portion of each well contained interferon-inducible protein of 10 kd (IP-10) which is a chemokine associated with inflammatory disease. The IP-10 chemokine was used because chronic lymphocytic leukemia cells express high levels of the CXCR3 chemokine receptor and are attracted by IP-10. The bar graph provides the numbers of cells crossing through the membranes into the lower well.

FIG. 5 illustrates that the number of peripheral blood leukocytes is reduced in mice by administration of 25 mg/kg (▴) etodolac or 100 mg/kg (▾) etodolac. Control mice (▪) receiving no etodolac did not exhibit lymphocyte depletion.

FIG. 6 illustrates that R-etodolac affects tumor cell movement without affecting their viability.

FIG. 6A is a photomicrograph of control, untreated chronic lymphocytic leukemia cells, illustrating that the normal pattern of chronic lymphocytic leukemia cellular growth is a large flat colony.

FIG. 6B illustrates that after incubation for 24 hours with 100 μM R-etodolac, the cells migrated toward each other, the diameter of the colonies was reduced, and multiple layers of cells were present in the colonies.

FIG. 6C illustrates that after incubation for 24 hours with 250 μM R-etodolac the cells migrated even more toward each other, the diameter of the colonies was even further reduced and even more layers of cells were present in the colonies. No effect on the total number of cells or their viability was observed.

FIG. 7 illustrates that R-etodolac affects the intracellular cytoskeleton in colon cancer HCT-116 cells so that the cells assume a rounded shape. The HCT-116 colon cancer cells were incubated for five hours with either no etodolac (FIG. 7A, control cells) or 500 μM R-etodolac (FIG. 7B, treated cells). The cells were then fixed and the intracellular actin filaments were stained with phalloidin conjugated to a fluorogenic dye (Alexa-green).

FIG. 7A. provides a photomicrograph of unstimulated, control cells where actin filaments are arranged in rigid stress fiber structures that provide a “stretched” cellular shape.

FIG. 7B provides a photomicrograph of R-etodolac treated cells where actin filaments do not form stress fibers and the cells assume a rounded shape.

### DETAILED DESCRIPTION OF THE INVENTION

According to the present invention, compounds that induce chemokinesis alter cell migration patterns, thereby interfering with immune and inflammatory responses and inhibiting the spread of malignant cells throughout the body. Contrary to conventional wisdom, induction of cellular chemokinesis by compounds identified through the present methods actually decrease the spread of malignant cancer cells and ameliorate the inflammatory response by causing leukocytes to be retained in the lymph nodes, spleen and other organs of the reticulo-endothelial system. Compounds identified by the present methods are useful for the treatment of inflammatory, allergic, and autoimmune diseases, for the prevention of allograft rejection, and for the therapy of lymphoproliferative diseases and cancer.

Definitions

According to the present invention, a chemoattractant molecule is any molecule known to one of skill in the art to induce cellular migration toward a higher concentration of that molecule.

As used herein, a chemorepulsive molecule is any molecule known to one of skill in the art to induce cellular migration toward a lower concentration of that molecule.

As used herein, chemokinesis is undirected cell migration. In contrast, chemotaxis is the directional migration of a cell in response to a gradient of chemoattractant or chemorepulsive molecules.

According to the present invention, a cognate receptor for a chemoattractant or chemorepulsive molecule is a receptor that can interact with the chemoattractant or the chemorepulsive molecule. Cognate, in general, refers to biomolecules that typically interact, for example, a receptor and its ligand. Examples of receptors contemplated by the present invention include the CXCR1 chemokine receptor, CXCR2 chemokine receptor, CXCR3 chemokine receptor, CXCR4 chemokine receptor, CXCR5 chemokine receptor, XCR1 chemokine receptor, CX₃CR1 chemokine receptor, CCR1 chemokine receptor, CCR2 chemokine receptor, CCR3 chemokine receptor, CCR4 chemokine receptor, CCR5 chemokine receptor, CCR6 chemokine receptor, CCR7 chemokine receptor, CCR8 chemokine receptor, CCR9 chemokine receptor, CCR10 chemokine receptor, the C5 a receptor, the arachidonate derivative leukotriene B₄ (LTB₄) receptor, the platelet activating factor (PAF) receptor, the formyl-met-leu-phe (fMLP) receptor, the neutrophil activating protein-1 (NAP-1) receptor, the interleukin 8 (IL-8) receptor, the platelet factor 4 receptor, the platelet basic protein receptor, the IP-10 receptor, the melanoma growth stimulating factor/GRO receptor and the like. Background information on these receptors is provided in Ziotnik et al., 12 Immunity 121-27 (2000) and Saunders et al., 4 DDT 80-92 (1999).

As used herein, a target cell is any cell type that can be cultured in vitro and that one of skill in the art can test by the present methods. In general, the term distinguishes one cell type from another. Hence, according to the present invention, the instant methods can be applied to specific cell types to the exclusion of other cell types. Examples of cell types contemplated by the present invention include basophils, eosinophils, endothelial cells, epithelial cells, fibroblasts, lymphocytes, macrophages, monocytes, neutrophils, neoplastic cells, polymorphonuclear leukocytes, colon cells, cancer or tumor cells, and the like. In one embodiment, the target cell is chronic lymphocytic leukemia cells which can be isolated from patients suffering from leukemia. In another embodiment, the cell line HL-60 (ATCC No. CCL 240) can be used as a source of mature myelocytes and neutrophils. This cell line can be maintained in logarithmic growth phase as a suspension culture at about 10⁶ cells/mL in RPMI 1640 medium (Mediatech Cellgrow, Fisher Scientific, Pittsburgh, Pa.) supplemented with 20% (volume by volume) fetal bovine serum (Hyclone Laboratories, Salt Lake City, Utah). The cells are differentiated into mature myelocytes and neutrophils by incubating the cells for 48 hours at 37° C. in media containing 1.5% (volume by volume) dimethylsulfoxide.

According to the present invention, the term “test compound” as used herein refers to a molecule being tested for the desired ability to promote or inhibit chemokinesis. In one embodiment, the methods of the present invention can be used to screen natural product or synthetic chemical libraries (e.g., peptide libraries) to identify novel chemokinetic inducers. In general, the test compound is used in an amount which is non-toxic to cultured cells. Preferably, the test compound can be formulated in a pharmaceutically effective amount and in a pharmaceutically acceptable manner so that it can be administered to a patient in need of treatment for an inflammatory disease, an allergy, an autoimmune disease, cancer, lymphoproliferative diseases, or to prevent allograft rejection.

Methods for Detecting Chemokinesis

The present invention provides methods for identifying compounds that induce chemokinesis which includes observing whether a test compound promotes random movement of a target cell or target cell population. The present invention is also directed to compounds isolated by these methods.

In one embodiment, compounds are tested to see whether they induce chemokinesis in a population of target cells by:

(a) contacting a population of target cells with an amount of a test compound in vitro; and

(b) determining the ability of the test compound to induce a chemokinetic response in the target cells.

According to the present invention, when the target cells are leukocytes, such a chemokinetic response is indicative of the ability of the test compound to reduce the level of circulating leukocytes in vivo, as in a mammal in need of such reduction.

An increased chemokinetic response is indicated, for example, when an increased number of cells pass from a chamber containing the cells in an appropriate medium through a microporous membrane. Thus, a chemokinetic response occurs when the number of cells passing through the microporous member following step (a) above, is greater than the number passing through said membrane prior to step (a). An increased chemokinetic response is also indicated, for example, when cellular migration through a microporous membrane is enhanced by a chemotaxin on the side of the membrane opposite a chamber containing the cells.

In order to observe whether a test compound can induce chemokinesis, cells must be exposed to or contacted by the test compound. The amount of test compound and the length of time that the cell is exposed to the test compound can vary. One of skill in the art can readily determine a suitable range of concentration for the test compound, for example, by generating a dose-response curve. Test cells can be cultured with a test compound for varying amounts of time before performing a chemokinesis test or the test compound can be added to the cell suspension during the chemokinesis test. For example, in one embodiment test cells are incubated overnight in medium containing the test compound at a concentration of 0 to 1000 μmol. A control set of cells incubated in medium containing no test compound is also prepared. An increase in chemokinesis as the concentration of test compound is increased indicates that the test compound can induce chemokinesis.

Any method known by one of skill in the art for observing the rate of cellular movement can be employed in the present methods to observe whether a compound can induce chemokinesis. Cellular chemokinesis can be measured by direct measurements of cell movement as assessed by passage across a semipermeable membrane such as is used in a Boyden chamber, or by migration of the cells on a slide through soft agarose. Cellular chemokinesis can be observed using a microscope, by forward scatter analysis, or using a cytofluorograph. Calcium uptake is also indicative of cellular chemokinesis and such uptake can be measured fluorometrically with a calcium sensitive dye. Activation of mitogen activated (MAP) kinase as measured by available enzyme assays or immunoblotting and a transition from an elongated, stretched cell shape to a rounder cell shape, as observed under a microscope is also used to detect induction of chemokinesis.

For example, according to the present invention, chemotaxis assay procedures can be adapted to test compounds for induction of chemokinesis. The currently used chemotaxis assay procedure derives from that originally developed by S. Boyden in 1962. See, S. Boyden, The Chemotactic Effect of Mixtures of Antibody and Antigen on Polymorphonuclear Leucocytes, J. Exp. Med. 115: pp. 453-466, 1962). For example, a suspension of cells can be placed in a chamber separated from a second chamber by a filter. After a predetermined period of time, the filter is removed and cells on the filter surface closest to the chamber containing the cell suspension are carefully removed. The remaining cells on the filter are then fixed and stained. Using a high power microscope, the filter is examined and the number of cells appearing on the side of the filter further from the chamber containing the cells is counted manually. A positive chemokinetic response is indicated when cells have migrated or “crawled” through the filter to the side further from the cell suspension. Because of the time required to do so, the entire filter is generally not examined. Rather, representative sample areas are examined and counted.

There are disadvantages to the filter cell-counting procedure. The examination and counting of the cells on the filter is time-consuming, tedious and expensive. It is also highly subjective because it necessarily involves the exercise of judgment in determining whether to count a cell that has only partially migrated across the filter. In addition, the time and expense associated with examining the entire filter necessitates that only representative areas, selected at random, be counted, thus rendering the results less accurate than would otherwise be the case if the entire filter were examined and counted. Moreover, this procedure requires that the cells be fixed which, of course, kills the cells. In order to determine a time-dependent relationship of the chemokinetic response, it is necessary to run multiple samples for each time point. When multiple samples with positive and negative controls, are needed to obtain reliable data, a single chemokinetic assay can produce dozens of filters, each of which needs to be individually examined and counted.

Alternatives to the Boyden assay have been proposed to overcome some of the above disadvantages. See generally, P. Wilkinson, Micropore Filter Methods for Leukocyte Chemotaxis, Methods in Enzymology, Vol. 162, (Academic Press, Inc. 1988), pp. 38-50. See also, Goodwin, U.S. Pat. No. 5,302,515; Guiruis et al., U.S. Pat. No. 4,912,057; Goodwin, U.S. Pat. No. 5,284,753; and Goodwin, U.S. Pat. No. 5,210,021.

The present invention can also employ non-destructive chemokinetic assay procedures.

For example, during or after exposure to a test compound, the cells can be labeled, the labeled cells placed in a first chamber and the movement of the labeled cells into a second chamber can be observed by following the movement of the label. The label can be any readily monitored label or reporter molecule known to one of skill in the art. For example, the label can be a dye, fluorescent moiety, or radioactive compound.

Any apparatus known to one of skill in the art can be used in the present chemokinetic assay procedures. For example, the apparatus can be any culture dish or plate in which a cell can be cultured and which can be adapted to have two chambers separated by filter or barrier that will allow test cells to migrate from one chamber to the other. In one embodiment, the apparatus can be a multi-well culture plate available from a variety of commercial sources. This type of apparatus is commonly employed to study the effects of chemical agents on cell growth. Each well of the culture plate can be provided with cup-shaped membrane insert adapted to fit inside and divide the well into two chambers. The size, shape and number of wells, inserts, and plate are not critical to this invention.

The cells can be placed in the cup-shaped membrane insert which is then placed into the well. For example, see U.S. Pat. No. 5,523,286 to McGlave et al. for a description of this type of apparatus. When present during testing, the test chemical can be present in both chambers of the well; otherwise the cells may be cultured with the test chemical for a time sufficient to induce chemokinesis. The membrane should be situated so as to be in contact with both fluid containing the cellular composition in the first chamber, and fluid in the second chamber. Except for added test molecules or cells, the fluids in the first and second chambers are preferably the same or substantially similar. When the cells exhibit a positive chemokinetic reaction, they will migrate or “crawl” from the chamber formed by the cup-shaped membrane insert through the pores in the membrane and into the second chamber.

In a preferred aspect, chemokinesis is detected or measured by determining data relative to a control or background level of migration into the second chamber using cells that have not been exposed to the test compound. An increased number of cells (or percentage of input cells, as the case may be) in the second chamber relative to the background level indicates chemokinesis has occurred.

The cells in the first and second chambers are maintained under conditions sufficient to allow cell migration. Such conditions generally are cell culture conditions. Any aqueous culture 25 medium known by one of skill in the art to permit cell migration can be used. For example, RPMI 1640 (e.g., from Gibco) (or L15) plus M199 (e.g., Gibco) (preferably in a 1:1 ratio) can be used. It is important to add some protein such as human serum albumin (HSA), bovine serum albumin (BSA), or fetal calf serum (FCS) to the fluid in both the first and second chambers, to a final concentration in the range of 0.25-1%; the same protein need not be present in both chambers. (Although not intending to be bound by any mechanism, Applicants believe that such proteins aid in the assay of the invention by increasing protein stability and inhibiting nonspecific sticking of cells.) Dilutions of test compounds and/or added chemoattractants are preferably carried out in fluid identical to that present in the chamber to which the compound or chemoattractant is to be added.

After placement of the cells in the first chamber, the apparatus is incubated to allow any chemokinesis of cells to take place. Incubation is carried out for a time period in the range of about 3-6 hours, and is most preferably done for 4 hours at about 37° C. In an embodiment where RPM11640 medium is employed in one or more of the chambers, incubation is preferably done at 5% Co₂; in an embodiment where L15 medium is employed, incubation at 5% CO₂ is not necessary since L15 can be used in room air.

The membrane used to divide the chambers of a chemokinetic apparatus may be of any convenient construction. The membrane is a microporous filter, of pore size in the range of about 3-8 microns, preferably 5-8 microns. The membrane can be made of a non-fibrous film of polyester, polycarbonate, polyethylene terephthalate, polylactic acid, nylon, nitrocellulose or the like. The thickness of the membrane is not critical to the invention. Membranes having a thickness in the range customarily used in the art are suitable for use herein. However, the membrane must have a plurality of pores disposed substantially perpendicular to the plane of the membrane surface which are sized to permit the migration of cells across the membrane. The diameter of the pores is not particularly critical and, to a large extent, depends upon the size of the cells being studied. Generally speaking, the pores must be of such diameter to prevent the cells from passively traversing the membrane while at the same permitting the cells to actively “crawl” through the membrane. It is readily within the skill of the ordinary artisan to determine the appropriate pore size for a particular chemokinetic assay without undue experimentation. Pores of suitable size can be provided in the film by any known process, such as atomic etching.

The space created between the cup-shaped membrane insert and the bottom of the well can vary. However, a space of about 1 mm between the bottom of well and cup-shaped membrane insert is generally sufficient to permit the free migration of cells across the membrane. To make the spaces in different wells of approximate uniform size, with legs, bosses, flange, radial projections and the like can be placed in the well before the cup-shaped membrane is inserted.

At predetermined periods, the quantum of cells that have migrated across the cup-shaped membrane insert are determined by observing the amount of label in the second chamber. When the label is a fluorescent label and membrane is opaque, the amount of label can be determined by measuring the radiation emitted by the labeled cells after exposure to a wavelength of light which will cause the label to fluoresce. Similarly, when the label is a dye and the membrane is opaque, the amount of label can be determined by observing the amount of light absorption by the dye at an appropriate wavelength. It will be understood by those skilled in the art that it is preferred that at least the chamber through which the stimulation and measurement of fluorescence and light absorption is substantially transparent to both the light being measured and the light needed to excite or to be absorbed by, the label. In the preferred embodiment, the apparatus is made of a clear, transparent material, such as polystyrene, polycarbonate, Lucite™, glass, and the like.

A target cell sample can be labeled with a fluorescent dye. The process of labeling cells with dyes is well known, as is the variety of fluorescent dyes that may be used for labeling particular cell types. See e.g. R. Haugland, Handbook of Fluorescent Probes and Research Chemicals, Molecular Probes, Inc. (1989). The cells are labeled before placing them in the chemokinesis chamber. This can be accomplished by any of various methods known in the art, for example, by fluorescent labeling of the cells, enzymatic labeling (e.g., via an enzyme-tagged antibody to a cell surface marker) or by another available method. In one embodiment, the cells are fluorescently labeled with fluorescein or a derivative thereof such as 2′,7′-bis-(2-carboxyethyl)-5(and 6)-carboxyfluorescein (BCECF) or calcein (Molecular Probes, Eugene, Ore.). For example, a fluorescent dye for use with an HL-60 cell line (ATCC No. CCL 240) is Di-I (Molecular Probes, Inc.; Eugene, Oreg.). For example, HL-60 cells can be incubated with 50 μM Di-I fluorescent dye (Molecular Probes, Inc., Eugene, Oreg.) at room temperature for 0.5-2 hours. The cells are then washed with Hanks' Balanced Salt Solution (“HBSS”) (Sigma Chemical Co., St. Louis, Mo.) and re-suspended in HBSS to achieve a cell concentration of 106 cells/mL. The fluorescence of 0.5 mL. of cell suspension can be measured in a Cytofluor™™ 2300 fluorescent plate reader (Millipore Corp., Marlborough, Mass.).

In another embodiment, compounds are tested to observe whether they cause homotypic aggregation of target cells and whether such homotypic aggregation by the target cells is blocked by a secretion inhibitor. Homotypic aggregation refers to aggregation by cells of the same type.

According to the present invention, any method for observing the movement, colony shape and aggregation of cells can be used to test for homotypic aggregation. For example, cells can be cultured in the presence of a test compound for a time and under conditions sufficient to permit cellular migration, if the compound is capable of inducing chemokinesis. Such conditions are generally normal cell culture conditions, but the culture conditions can be modified as needed by one of skill in the art. Homotypic aggregation can be determined by observing the live cells under a microscope. If the cells form large, flat colonies, no homotypic aggregation is indicated. However, homotypic aggregation is indicated when the cells migrate toward one another and form smaller, multi-layered colonies in which the cells crowd on top of one another. Cell aggregation can also be detected by observing cell shape changes characteristic of migrating cells, for example, by observing cytoskeletal reorganization as assessed fluorometrically by the binding of actin specific dyes such as phalloidin.

Any secretion inhibitor available to one of skill in the art may be used to test whether cellular aggregation is blocked. An example of a secretion inhibitor contemplated by the present invention is brefeldin A.

Formation of rounder cell shapes from stretched cellular shapes is also indicative of chemokinesis induction. The pattern of actin filaments in cells, detected for example by phalloidin conjugated to a fluorogenic dye, can be used to detect changes in cell shape indicative of chemokinesis. For example, cells with stretched shapes where the actin forms rigid, elongated stress fibers are indicative of no chemokinesis induction. When chemokinesis is induced, the actin fibers become rounded and the cells become more spherical.

In another embodiment, compounds are tested to see whether they stimulate a target cell, which has a cognate receptor for a chemotactic molecule, to produce that chemotactic molecule. 25 Chemotactic molecules which may be detected include, for example, GRO-α, GRO-β, MGSA-α, MGSA-β, MGSA-γ, PF₄, ENA-78, GCP-2, NAP-2, IL-8, IP10, IL-8, I-309, I-TAC, SDF-1, BLC, BCA-1, BRAK, bolekine, ELC, LKTN-1 lymphotactin, SCM-1β, fractalkine, I-309, MIG, MCP-1, MCAF, MIP-1α, MIP-3α, LD7α, MIP-1β, RANTES, MCP-3, MCP-2, eotaxin, MCP-4, MCP-5, HCC-1, HCC-2, Lkn-1, HCC-4, LARC, LEC, TARC, DC-CK1, PARC AMAC-1, MIP-2β, ELC, exodus-3, MIP-3β, ARC, exodus-1, 6Ckine, SLC, exodus-2, MDC, STCP-1, MPIF-1, MPIF-2, Eotaxin-2, TECK, Eotaxin-3, CTACK, ILC and the like. Background information on these chemotactic molecules is provided in Ziotnik et al., 12 Immunity 121-27 (2000) and Saunders et al., 4 DDT 80-92 (1999).

According to the present invention, any method known to one of skill in the art for detecting production of a chemotactic molecule can be used. For example, a chemotactic molecule can be detected by ELISA using commercially available kits (e.g. from R&D Systems), by detection of chemotactic activity in the test medium when used with another target cell type, by SDS-PAGE analysis, by thin layer chromatography, by HPLC, by use of an antibody known to be specific for the chemotactic molecule and other methods available to one of skill in the art.

The chemokinesis assay procedures described above provide methods for identifying compounds that can induce chemokinesis. In general, any test compound that induces cell migration, cell movement, calcium uptake, MAP kinase activation, cell shape changes, homotypic aggregation or the production of a chemoattractant is a compound that can induce chemokinesis. These compounds can be characterized further by the following procedures.

Characterization of Compounds that can Induce Chemokinesis

According to the present invention, induction of cellular chemokinesis by compounds identified through the present methods can decrease the spread of cancer cells in vitro and in vivo and thereby inhibit metastatic cancer. Cellular chemokinesis induced by the present compounds can also ameliorate the inflammatory response by causing leukocytes to be retained in the lymph nodes, spleen and other organs of the reticulo-endothelial system. Compounds identified by the present methods are therefore useful for the treatment of inflammatory, allergic, arthritic, asthmatic and autoimmune diseases, for the prevention of allograft or tissue rejection, and for the therapy of lymphoproliferative diseases and cancer.

To further examine the test compound in vivo and determine optimal conditions for inducing chemokinesis for treatment of these diseases, the compound can be administered to an animal, e.g. a rodent, and its effects observed by any procedure available to one of skill in the art. In general, a test compound is efficacious for treating inflammation, allergies, arthritis, autoimmune disease, tissue or transplant rejection, lymphoproliferative diseases or cancer if the level of infiltrating leukocytes is reduced after administration of the compound in the affected tissue relative to the level of circulating leukocytes in the blood. Hence, to evaluate the efficacy of a compound and determine a pharmaceutically effective dosage for that compound, blood may be withdrawn and a small amount of the affected tissue may be excised from a mammal after administration of an amount of the compound. If the level of infiltrating leukocytes is reduced in the excised tissue relative to the level of leukocytes in the blood, the compound has efficacy for treating the disease. In another embodiment, the level of infiltrating leukocytes in a tissue excised from a mammal which has not received the test compound is compared to the level a tissue excised from a mammal which has received the test compound. Another method for evaluating a test compound and determining an efficacious dosage is to determine what amount of compound is needed to induce leukocytes to accumulate in the spleen, lymph nodes and other organs of the reticulo-endothelial system. One of skill in the art can readily adapt these procedures to evaluate a test compound and determine an efficacious dosage.

The chemokinesis assay is also useful for identifying the appropriate amount or dosage to achieve the desired chemokinetic effect in target cells in vitro and to estimate the dosage required in vivo. The in vitro dosage may be similar to, or somewhat different from, the dose of the identified compound used for therapeutic purposes.

According to the present invention, compounds identified by the present methods can affect a specific cell type to the exclusion of other cell types. Hence, compounds of the present invention can specifically induce chemokinesis in lymphocytes but not neutrophils or eosinophils. Moreover, the present compounds can induce chemokinesis in specific types of lymphocytes, for example, in CD8 T cells but not in CD4 T cells. Compounds isolated according to the present methods can induce chemokinesis in one or more of the following cell types, all lymphocytes, CD8 T cells, B cells, CD4 T cells, all phagocytic lymphocytes, neutrophils, basophils, eosinophils, monocytes and the like. Induction of chemokinesis in CD8 T cell lymphocytes is beneficial for treating transplant or tissue rejection. Induction of chemokinesis in CD4 T cell lymphocytes is beneficial for treating autoimmune diseases and allergies. Induction of chemokinesis in neutrophils is beneficial for acute inflammation. Induction of chemokinesis in monocytes is beneficial for treating chronic inflammation. Induction of chemokinesis in basophils or eosinophils is beneficial for treating allergic inflammations and asthma.

For example, the test compound can be injected into the skin, subcutaneous tissues, lungs or other organs of a rodent with or without a chemotactic irritant such as carrageenan, oil, etc. After an interval of time ranging from one or more hours to several days, the rodent can be sacrificed and the accumulation of leukocytes within the lymph nodes, spleen and other organs of the reticulo-endothelial system can be determined histologically, by viewing these tissues under a microscope or by enzymatic assay for specific enzymes associated with a given leukocyte cell type. Alternatively, leukocytes can be pre-labeled with a radioisotope or fluorochrome and followed with a detector. Eosinophils can be identified by their characteristic histologic appearance, by measurement of their unique enzymes and by eosinophil-specific antibodies.

According to the present invention, test compounds that induce chemokinesis can inhibit metastatic cancer in vivo. Procedures available to one of skill in the art can be used to characterize test compounds in this regard. For example, according to the present invention, test compounds that induce chemokinesis can reduce the survival of chronic lymphocytic leukemia cells (B-CLL cells) in vivo. The test compound can be administered to patients suffering from chronic lymphocytic leukemia in a pharmaceutically effective amount. B-CLL cells are isolated just before administration of the test compound, and at various time periods after and/or during the administration of the test compound. B-CLL cells recovered from patients are then cultured under the appropriate conditions. The apoptosis rates are measured in the B-CLL cell populations isolated before and after test compound administration. In one experiment, cells isolated from five out of six patients, after in vivo etodolac challenge displayed more ex vivo spontaneous apoptosis than cells obtained just before etodolac therapy (Table 1 below).

Also according to the present invention, test compounds that induce chemokinesis can inhibit the inflammatory response. The inflammatory response can be observed by any method available to one of skill in the art. In one embodiment, the test compound can inhibit inflammation mediated by eosinophils. For example, eosinophil-associated inflammatory conditions include bronchial asthma, and other conditions disclosed in U.S. Pat. No. 5, 837,713.

For example, the anti-inflammatory effect of selected compounds of this invention can be compared to those of Triclosan™ and hydrocortisone in a modified 12-tetradecanoyl 13-phorbol acetate (TPA) mouse ear inflammation assay. TPA can be used as an inflammogen rather than croton oil because TPA gives a well characterized inflammatory response at very low concentrations. The use of this mouse model has been shown to reflect the clinical parameters characteristic of inflammatory responses in humans and predictive of the effectiveness of therapeutic agents in patients (see, e.g., Kimura et al., 1995, Biological and Pharmaceutical Bull. 18:1617-1619; Rao et al., J. Lipid Mediators & Cell Signaling 10:213-228; Fretland et al., Inflammation 19:333-346). This model can therefore be used to study the pharmacokinetics, clinical efficacy and adverse side effects of anti-inflammatory agents.

To quantitate inflammation, ear punches from treated animals can be used to measure increased ear mass (edema) and myeloperoxidase (MPO) activity. For the inhibition studies, ear biopsies can be weighed six hours after treatment with TPA and the simultaneous application of the compound of the present invention. Following weight determination, the biopsies can be frozen and subsequently used to measure inhibition of MPO activity, which is one method of estimating polymorphonuclear (PMN) lymphocyte activity in the affected area. Percent inhibition of edema can be calculated as [c−t]/c×100, where c and t are increases in ear weight in control and treated mice, respectively. A dose-response curve for TPA-induced mouse ear edema can be generated in order to determine the concentration of TPA to be employed in the inflammatory inhibition studies.

Also according to the present invention, test compounds that induce chemokinesis can inhibit cell, tissue or organ rejection by a mammalian recipient. Cell, tissue and organ rejection by a mammal can be evaluated by any method available to one of skill in the art. For example, models of cell, organ and tissue rejection are set forth in U.S. Pat. No. 6,106,834. In one embodiment, the test compound can inhibit edema or inflammation due to transfusion, hemodialysis or ischemia/reperfusion injury.

For example, tissue rejection can be evaluated by the skin graft method previously described by D. Steinmuller, Skin Grafting. Surgical Techniques in Immunology, Methods Enzymol. 108, 20 (1984). Briefly, a tailskin from an 8-12 week old male B 10.Br mouse is removed and stored in cold saline. Male C57BL/10 mice are anesthetized, and their backs are shaved. The backs are scrubbed with alcohol, and a 1 cm² piece of skin is removed. A similar size piece of skin is cut from the tailskin of the B 10.Br mouse and placed in the excised area on the C57BL/10 animal's back. A petroleum jelly coated bandage is placed over the graft and held in place by a bandage. Test compounds are administered in a pharmaceutically effective amount beginning on the day of skin grafting and continuing until the end of the test period or until transplant rejection. Bandages are left in place until 8 days post grafting. At that time they are removed, and the grafts are observed daily for signs of rejection. Rejection is determined by complete blackening or scabbing of the grafted skin. This and other available procedures can readily adapted by one of skill in the art to permit characterization of the inhibitory properties of test compound and the degree to which they can inhibit edema or inflammation from transfusion, hemodialysis or ischemia/reperfusion injury.

According to the present invention, test compounds that induce chemokinesis can inhibit the autoimmune response. The autoimmune response can be observed by any method available to one of skill in the art.

For example, autoimmune responses can be monitored using the methods provided in U.S. Pat. No. 6,197,596 to Newkirk. In general, Newkirk observed that serum clusterin levels are significantly decreased in patients with systemic lupus erythematosus (SLE) and the level of clusterin correlates inversely with disease activity. Low clusterin levels are associated with the skin lesions, loss of hair, proteinuria and the presence of arthritis. According to Newkirk, low levels of clusterin are detrimental to patients with SLE and appear to contribute to the disease pathogenesis.

Serum clusterin can be measured by a modified capture ELISA (Hogasen, K. et al., J. Immunol. Meth. 160:107-115 (1993)). Clusterin is captured onto high binding ELISA plates (either EIA plus, ICN, Montreal, QC or plate F, Greiner, BellCo, Vineland N.J.) from the purified standard (dilution curve was established using human clusterin, Quidel, San Diego, Calif.) or sera diluted from 1:100 to 1:8000 (most frequent dilution used to calculate amount of clusterin was 1:4000) as appropriate, in PBS, 0.2% Tween™ 20. After an overnight incubation at 4° C., the plates are washed with PBS, 0.1% Tween™ 20.

Monoclonal anti-clusterin antibody (SP40,40/G7 mAb, Quidel) diluted 1:10,000 in PBS, 0.1% Tween™ 20 is added and the plates were incubated for 1 hr at 37.degree. C. After washing, HRP-conjugated F(ab′)₂ fragments of sheep anti-mouse IgG antibodies (Jackson, BioCan, Mississauga, ON) are used (diluted 1:20,000) to detect the bound anti-clusterin antibodies. After a 1 hr incubation at 37° C., the plate is washed and the substrate added (o-phenylene-diamine) for 30 minutes at 25° C. The reaction was terminated with 4 M H₂ SO₄, and the optical density at 492 (reference 690) was measured, using an ELISA plate reader (SLT LabInstruments, Fisher, Montreal, QC). Since there is a saturation level to the plates, clusterin is calculated from that dilutions of serum or plasma where with a doubling dilution there was a 2-fold change in O.D. These and other available procedures can readily adapted by one of skill in the art to permit characterization of the inhibitory properties of test compound and the degree to which they can inhibit an autoimmune response.

In one embodiment, etodolac, or an analog thereof, is used in the therapeutic methods of the present invention. Etodolac can be prepared by methods available to one of skill in the art. Etodolac analogs include those described in U.S. Pat. No. 3,843,681 and U.S. patent application Ser. No. 09/634,207, which are incorporated by reference.

### EXAMPLES

### Example 1

### Materials and Methods

Patients, Cell Isolation and Viability Assays

Written informed consent was obtained to procure peripheral blood from all patients and with normal healthy volunteers. Patients had to have B-CLL according to National Cancer Institute (NCI) criteria of any Rai stage. Criteria for requiring therapy were as follows: disease-related symptoms, anemia and/or thrombocytopenia, bulky lymphadenopathy, and/or clinically relevant splenomegaly. Mononuclear cells were isolated by gradient centrifugation through Ficoll-Paque. Cells were cultured at 2×10⁶ per mL in RPMI 1640 with 20% autologous plasma and antibiotics in 96-well plates without or with various concentrations of etodolac (racemic, S-etodolac, or R-etodolac). At the indicated times, viability assays were performed by erythrosin B dye exclusion.

Separation of Etodolac Enantiomers

Etodolac isomers were separated by fractional crystallization by a modification of the procedure of Becker-Scharfenkamp and Blaschke, 621 J. Chromtgr. Biomed. Applns 199-207 (1993). Briefly, pharmaceutical grade tablets of racemic etodolac (6×400 mg) were crushed in a mortar and pestle and extracted with hot ethyl acetate (2×50 mL) and filtered. Evaporation of the filtrate gave 1.52 g of white powder (68% recovery). This material was dissolved in 2-propanol (10 mL) and S- or R-phenylethylamine was added. The clear solution was allowed to stand at 4° C. for several days. The resulting colorless needles were collected and recrystallized from 2-propanol two times. The salt product was decomposed by adding to ice cold 10% sulfuric acid and extracting with ethyl acetate. After evaporation, the syrupy residue of R- or S-etodolac was crystallized from methanol-water. The enantiomeric purity of each product was at least 97% as assayed by HPLC on a chiral column (AGP, ChromTech, Sweden).

Protein Expression Assays.

Washed B-CLL cells were lysed in Lysis buffer (25 mM Tris, pH 7.4, 150 mM KCl, 5 mM EDTA, 1% Nonidet P-40, 0.5% sodium deoxycholate, 0.1% SDS, 1 μg/ml aprotinin, 1 μg/ml leupeptin, 1 mM phenylmethanesulfonyl fluoride, 1 mM sodium orthovanadate, 1 mm sodium fluoride). Lysates were centrifuged at 15,000× g for 10 min to remove nuclei and the protein content of supernatants was measured using a modified Coomassie blue assay (Pierce, Rockford, Ill.). Proteins were resolved at 125 V on 14% and 4-20% Tris-gly pre-cast gels (Novex, San Diego) and electrophoretically transferred to 0.2 μm polyvinylidene fluoride (PVDF) membranes (Millipore, Bedford, Mass.) for 2 hours at 125 V. Membranes were blocked overnight in I-Block blocking buffer (Tropix, Bedford, Mass.). Blots were probed with polyclonal antibodies anti-Mcl-1, anti-Bcl-2, monoclonal anti-Bag-1, monoclonal anti-XIAP (Transduction Laboratories Inc.), monoclonal anti-PARP (gift of N.A. Berger), or monoclonal anti-PPAR-γ (Santa Cruz Laboratories, Calif.) antibody, followed by secondary antibody consisted of horseradish peroxidase (HRP)-conjugated goat anti-rabbit (Mcl-1) or anti-mouse (PPAR-γ) IgG. Detection was performed by an enhanced chemiluminescence (ECL, Amersham) method, followed by calorimetric detection, using SG substrate. The X-ray films were scanned, acquired in Adobe Systems Photoshop and analyzed with NIH Image Software.

Migration/chemotaxis Assay

Cell migration was measured in a 24-well modified Boyden chamber (Transwell, Corning-Costar, N.Y.). The recombinant human IP-10 chemokine (R&D Systems, McKinley Place, Nebr.) was diluted in RPMI-1640 medium at 200 ng/ml, and used to evaluate the chemotactic properties of lymphocytes from B-CLL patients. Polycarbonate membranes with pore size of 3 μm were used. A total of 600 mL of chemokines or control medium was added to the bottom wells, and 100 mL of 2 to 5.0×10⁶ cells/ml cells resuspended in RPMI-1640 were added to the top wells. The chamber was incubated at 37° C. with 5% CO₂ for 2 hours. The membranes were then removed, and the cells present on the bottom well were quantified by flow cytometry. For cell quantification, a fixed acquisition time of 30 seconds was used per sample, and beads were run during each experiment to ensure a reproducible acquisition. All assays were performed in triplicate.

B-Chronic lymphocytic leukemia (B-CLL) cells were pre-incubated in the presence of various concentrations of etodolac for 16 hours, prior to loading in the upper part of a modified Boyden chamber with pore size of 3 μm. The lower reservoir contained either medium alone, or the CXCR3-binding chemokine IFN-inducible protein 10 (IP-10, 200 ng/ml). After 2 hours of incubation, the cells that traveled through the polycarbonate membrane were enumerated by flow cytometry.

### Example 2

### Etodolac Induces Chemokinesis in Chronic Lymphocytic Leukemia Cells

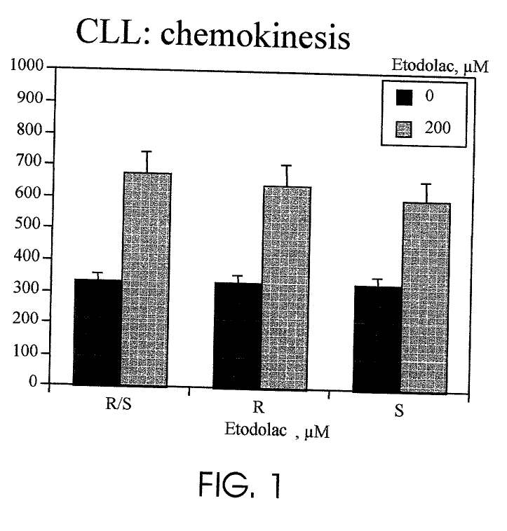

Chronic lymphocytic leukemia cells were incubated overnight in medium containing a racemic mixture of etodolac, the R-enantiomer of etodolac or the S-enantiomer of etodolac at 200 μmol concentration. Control cells were incubated in the same medium without etodolac. The cells were then added to the upper wells of chemotaxis chambers divided by a 5 to 8 micron membrane. No etodolac was present in the lower portion of each well. FIG. 1 provides a graph of the number of chronic lymphocytic leukemia cells crossing through the membranes into the lower well. As shown in FIG. 1, more cells migrated through the membrane after exposure to etodolac (green bars) than when no etodolac was present (blue bars). The enantiomeric form of etodolac did not significantly influence the migration of cells.

### Example 3

### Etodolac Induces Chemokinesis in Peripheral Blood Lymphocytes

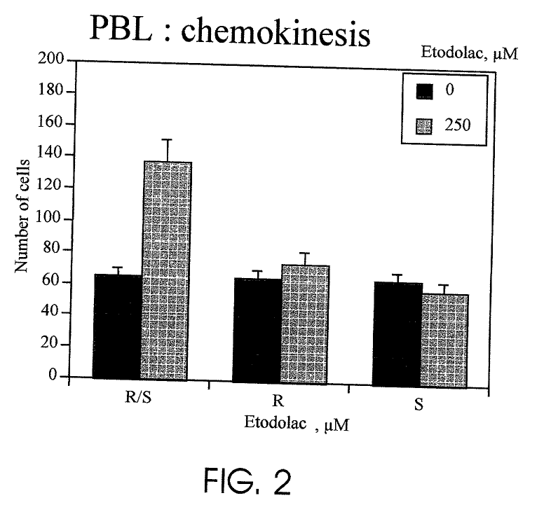

Peripheral blood lymphocytes were incubated overnight in medium containing a racemic mixture of etodolac, the R-enantiomer of etodolac or the S-enantiomer of etodolac at 250 μM concentration. Control cells were incubated in the same medium without etodolac. The cells were then added to the upper wells of chemotaxis chambers divided by a 5 to 8 micron membrane. No etodolac was present in the lower portion of each well. FIG. 2 provides a graph of the number of peripheral blood lymphocytes crossing through the membranes into the lower well. As shown in FIG. 2, more cells migrated through the membrane after exposure to the racemic mixture of etodolac (green bar) than when no etodolac was present (blue bar), however the separate enantiomers of etodolac did not induce substantial chemokinesis.

### Example 4

### Etodolac Induces Chemokinesis in Chronic Lymphocytic Leukemia Cells

The B-CLL patients on etodolac therapy did not have clinically obvious changes in lymphadenopathy. To determine if etodolac could alter the distribution of B-CLL between the blood and lymphoid organs, the effects of the drug on CXCR3-dependent chemotaxis were tested. Chronic lymphocytic leukemia (B-CLL) cells from patients were incubated overnight in medium containing a racemic mixture of etodolac, the R-enantiomer of etodolac or the S-enantiomer of etodolac at concentrations varying between 12.5 μM to 500 μM. The cells were then added to the upper wells of chemotaxis chambers divided by a 5 to 8 micron membrane. The lower portion of each well contained interferon-inducible protein of 10 kd (IP-10) which is a chemokine associated with inflammatory disease. The IP-10 chemokine was used because B-CLL cells express high levels of the CXCR3 chemokine receptor and are attracted by IP-10.

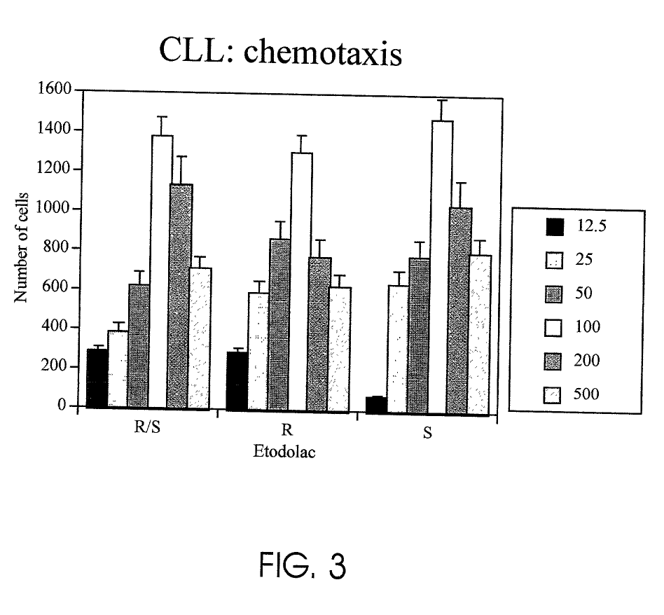

In all B-CLL patients tested (n=4), etodolac increased the chemotactic response of B-CLL cells to IP-10 (FIG. 3). Enhanced chemotaxis was observed in B-CLL cells incubated with 50 mM etodolac, well below the plasma concentration expected in patients receiving 800 mg/day of the drug. At concentrations of 500 μM and above, chemotaxis decreased, due to a loss of cell viability. Not only the racemic (R/S) mixture of etodolac, but also the purified R and S enantiomers, enhanced B-CLL chemotaxis. Stimulation of chemotaxis was not due to increased expression of the CXCR receptor, as assessed by antibody staining (data not shown).

### Example 5

### Etodolac Induces Chemokinesis in Peripheral Blood Lymphocytes

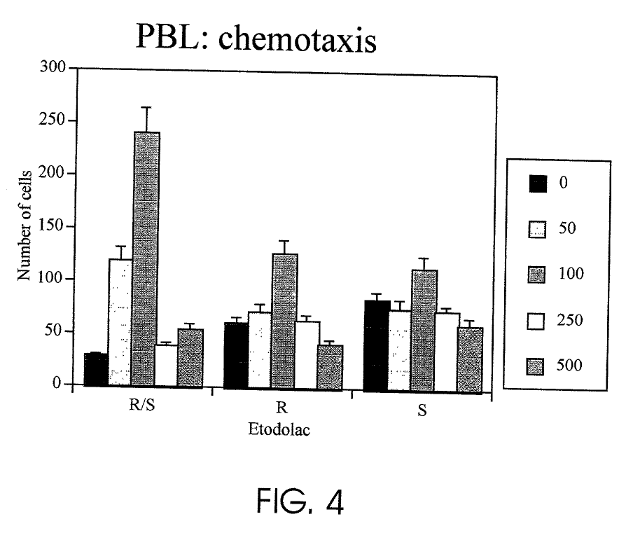

Peripheral blood lymphocytes were incubated overnight in medium containing a racemic mixture of etodolac, the R-enantiomer of etodolac or the S-enantiomer of etodolac at concentrations varying between 0 μM to 500 μM. The cells were then added to the upper wells of chemotaxis chambers divided by a 5 to 8 micron membrane. The lower portion of each well contained interferon-inducible protein of 10 kd (IP-10) which is a chemokine associated with inflammatory disease. FIG. 4 provides a graph of the number of peripheral blood lymphocytes crossing through the membranes into the lower wells. As shown in FIG. 4, the number of cells migrating through the membrane after exposure to racemic form of etodolac increased with etodolac concentration until the etodolac concentration reached 100 μM. At concentrations of 250 μM and above, chemotaxis decreased, due to a loss of cell viability. The separate enantiomeric forms of etodolac caused less cell migration than did the racemic mixture of etodolac.

### Example 6

### Etodolac Induces Lymphocyte Depletion in Mice

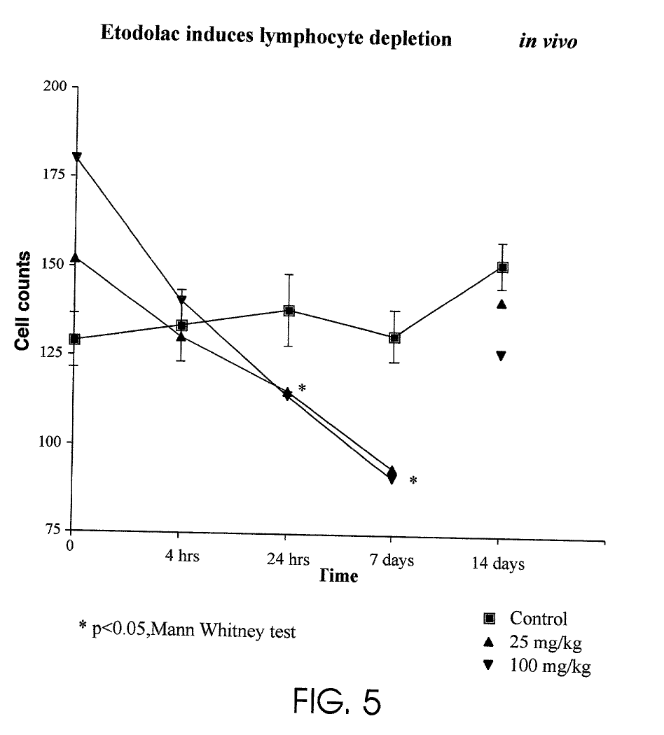

Normal mice were given 25 mg/kg etodolac or 100 mg/kg etodolac by gastric lavage. Control mice received no etodolac. Blood was drawn from each mouse at 4 hr, 24 hr, 158 hr and 14 days after etodolac administration and peripheral blood leukocyte counts were done. FIG. 5 shows that the number of peripheral blood leukocytes is reduced in mice by administration of 25 mg/kg (▴) etodolac or 100 mg/kg (▾) etodolac. Control mice (▪) receiving no etodolac did not exhibit lymphocyte depletion.

### Example 7

### Etodolac Induces B-CLL Cells to Migrate

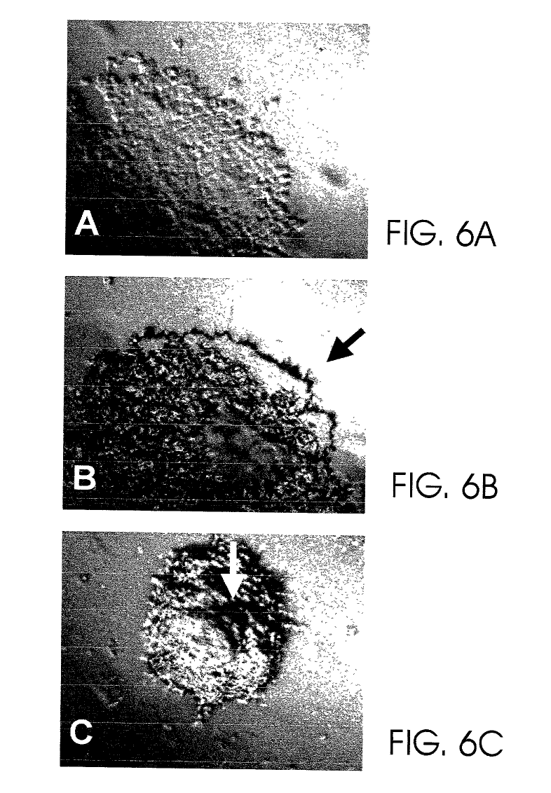

Chronic lymphocytic leukemia (B-CLL) cells were incubated for 24 hr in medium containing the R-enantiomer of etodolac at 0.0 μM, 100 μM and 250 μM. concentrations. The cell morphology was observed under light microscopy. Control cells incubated without etodolac grew in large, flat colonies (FIG. 6A). However, after incubation for 24 hours with either 100 μM or 250 μM. R-etodolac the cells migrated toward each other and the diameter of the colonies was reduced (FIGS. 6B and 6C). No effect on the total number of cells or their viability was observed in the same experimental conditions, at the indicated concentration. These results indicate that R-etodolac affects tumor cell movement, and cellular aggregation, at the concentrations tested.

### Example 8

### Etodolac Induces Morphological Changes in Colon Cancer Cells

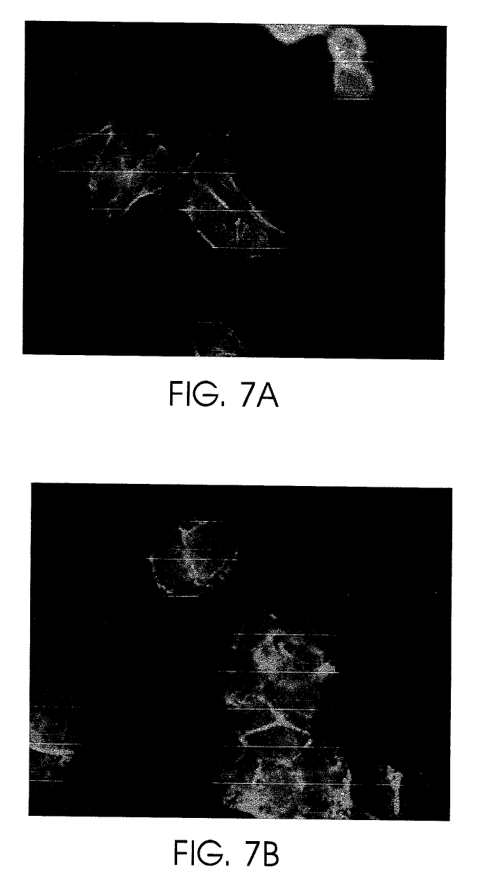

Colon cancer cells (HCT-116) were incubated for 5 hours with either no etodolac (control cells) or 500 μM. R-etodolac (test cells). The cells were then fixed and the intracellular actin filaments were stained with phalloidan conjugated to a fluorogenic dye (Alexa-green). The control cells (FIG. 7A) had actin filaments arranged in rigid structures (stress fibers) that confer the “stretched” shape often seen in normal cells. In R-etodolac treated cells (FIG. 7B), the actin filaments are modified, no conventional stress fibers are observed and the cells assume a rounded shape. These data indicate that R-etodolac affects the intracellular cytoskeletal components of tumor cells.

### Example 9

### Etodolac Induces a Transient Reduction of Lymphocyte Counts in B-CLL Patients.

To determine if etodolac could reduce B-CLL survival in vivo, six B-CLL patients were enrolled in a first step challenge assay for etodolac sensitivity. Each patient received one 400 mg etodolac tablet, and a second tablet 12 hours later. B-CLL cells were isolated just before the first tablet, and 12 hours after the second. B-CLL cells recovered from patients were then cultured with 20% autologous plasma and apoptosis rates were measured, either before or after etodolac treatment. In 5 out of 6 patients, cells isolated after in vivo etodolac challenge displayed more ex vivo spontaneous apoptosis than cells obtained just before etodolac therapy (Table 1). The five etodolac-sensitive patients were then enrolled in a second-step clinical trial in which they took etodolac for one month (400 mg bid). When etodolac treatment was interrupted, B-CLL counts rebounded to pre-treatment levels. Two of the patients subsequently were retreated, and the circulating lymphocyte counts dropped again by almost 50% over one month (results not shown).

Isolated B-CLL cells before and after etodolac treatment (400 mg bid) were cultured in RPMI-1640 with 20% autologous plasma, collected either before or after therapy. After 72 hour of incubation, viability assays were performed by erythrosin B staining. Numbers indicate the % of viable cells. Note that B-CLL cells die spontaneously over time and that etodolac increased the cell death in 5 out of 6 patients.

TABLE 1

| Patient | Viability Before | Viability After | % Increased cell death |
|---|---|---|---|
| #1 | 90 ±± 8 | 78 ±± 5 | 13 |
| #2 | 47 ±± 5 | 20 ±± 3 | 57 |
| #3 | 75 ±± 6 | 31 ±± 5 | 69 |
| #4 | 96 ±± 11 | 96 ±± 9 | 0 |
| #5 | 50 ±± 6 | 33 ±± 4 | 34 |
| #6 | 68 ±± 4 | 40 ±± 6 | 41 |

According to the present invention, B-CLL cells exposed to etodolac are sequestered within lymphoid organs, where chemokine concentrations are likely to be higher than in the plasma. Moreover, while early clearance of B-CLL cells from circulation may result mainly from a change in cell distribution, the impaired survival observed after in vivo treatment will be reflected eventually by reductions in tumor burden.

All cited art and patents cited herein are incorporated by reference herein as though fully set forth.

What is claimed:

1. A method to determine the ability of a test compound to induce chemokinesis in a population of leukocytes comprising:
(a) contacting the population of leukocytes with an amount of said test compound in vitro; and
(b) determining the ability of the compound to induce a chemokinetic response in said leukocytes; wherein the chemokinetic response is indicative of the ability of the compound to reduce the level of circulating leukocytes in vivo.

2. The method of claim 1 wherein said response enhances the ability of the cells to exhibit chemotaxis.

3. The method of claim 1 wherein the leukocytes are lymphocytes.

4. The method of claim 1 wherein the chemokinetic response comprises an increase in the random movement of said cells.

5. The method of claim 1 wherein the chemokinetic response comprises the ability to stimulate release from said cells of a chemotactic molecule that binds to a receptor on the cells for said molecule.

6. The method of claim 1 wherein the chemokinetic response comprises homotypic aggregation of the target cells, wherein said aggregation can be blocked by a secretion inhibitor.

7. The method of claim 1, 2 or 3 wherein the cells are human cells.

8. The method of claim 1, 2 or 3 wherein the leukocytes are lymphocytes or monocytes.

9. The method of claim 8 wherein the lymphocytes are B lymphocytes.

10. The method of claim 1, 2 or 3 wherein the leukocytes are neutrophils, eosinophils, or basophils.

11. The method of claim 2 wherein the chemotaxis is induced by GRO-α, GRO-β, MGSA-α, MGSA-β, MGSA-γ, PF₄, ENA-78, GCP-2, NAP-2, IL-8, IP10, IL-8, I-309, I-TAC, SDF-1, BLC, BCA-1, BRAK, bolekine, ELC, LKTN-1 lymphotactin, SCM-1β, fractalkine, I-309, MIG, MCP-1, MCAF, MIP-1α, MIP-3α, LD7α, MIP-1β, RANTES, MCP-3, MCP-2, eotaxin, MCP-4, MCP-5, HCC-1, HCC-2, Lkn-1, HCC-4, LARC, LEC, TARC, DC-CK1, PARC AMAC-1, MIP-2β, ELC, exodus-3, MIP-3β, ARC, exodus-1, 6Ckine, SLC, exodus-2, MDC, STCP-1, MPIF-1, MPIF-2, Eotaxin-2, TECK, Eotaxin-3, or CTACK, ILC.

12. The method of claim 2 wherein the chemotaxis is induced by IP-10.

13. The method of claim 1 wherein the cells are neoplastic.

14. The method of claim 1 or 2 which further comprises evaluating the ability of the compound to inhibit metastatic cancer in vivo.

15. The method of claim 1 which further comprises evaluating the ability of the compound to inhibit a deleterious inflammatory response.

16. The method of claim 1 further comprising evaluating the ability of the compound to inhibit cell, tissue or organ rejection by a mammalian recipient.

17. The method of claim 1 which further comprises evaluating the ability of the compound to inhibit an autoimmune response.

18. The method of claim 17 which further comprises evaluating the ability of the compound to inhibit inflammation mediated by eosinophils.

19. The method of claim 17 which further comprises the ability of the compound to inhibit edema or inflammation due to transfusion, hemodialysis or ischemia/reperfusion injury.

20. The method of claim 1 wherein an increased chemokinetic response is indicated by an increased number of cells passing from a chamber containing said cells through a microporous membrane, following step (a), over the number passing through said membrane prior to step (a).

21. The method of claim 1 wherein an increased chemokinetic response following step (a) causes increased migration of the cells through a microporous membrane, wherein said migration is enhanced by a chemotaxin on the side of the membrane opposite a chamber containing the cells.

22. A method to determine the ability of a test compound to induce chemokinesis in a population of target cells which comprises:
(a) contacting the population of target cells with an amount of said test compound in vitro; and
(b) observing whether said target cells produce a chemotactic molecule; wherein said target cell has a cognate receptor for said chemotactic molecule.

23. A method to determine the ability of a test compound to induce chemokinesis in a population of target cells which comprises:
(a) contacting the population of target cells with an amount of said test compound in vitro; and
(b) observing whether said target cells homotypically aggregate.

24. A method to determine the ability of a test compound to induce chemokinesis in a population of target cells which comprises:
(a) contacting the population of target cells with an amount of said test compound in vitro; and
(b) observing whether actin filaments in said target cells form stress fibers; wherein a positive chemokinetic response is indicated when said target cell do not form stress fibers.

25. The method of claim 22, 23 or 24 wherein said target cells are basophils, eosinophils, endothelial cells, epithelial cells, fibroblasts, lymphocytes, macrophages, monocytes, neutrophils, neoplastic cells, polymorphonuclear leukocytes, colon cells or tumor cells.

26. The method of claim 22 wherein said cognate receptor for said chemotactic molecule is a CXCR1 chemokine receptor, CXCR2 chemokine receptor, CXCR3 chemokine receptor, CXCR4 chemokine receptor, CXCR5 chemokine receptor, XCR1 chemokine receptor, CX₃CR1 chemokine receptor, CCR1 chemokine receptor, CCR2 chemokine receptor, CCR3 chemokine receptor, CCR4 chemokine receptor, CCR5 chemokine receptor, CCR6 chemokine receptor, CCR7 chemokine receptor, CCR8 chemokine receptor, CCR9 chemokine receptor, CCR10 chemokine receptor, C5a receptor, arachidonate derivative leukotriene B₄ receptor, platelet activating factor receptor, formyl-met-leu-phe receptor, neutrophil activating protein-1 receptor, interleukin 8 receptor, platelet factor 4 receptor, platelet basic protein receptor, or melanoma growth stimulating factor/GRO receptor.

27. A compound identified by the method of claim 1, 22, 23 or 24.

28. A therapeutic method for treating a condition ameliorated by induction of chemokinesis in a specific cell type of a mammal which comprises systemically administering to a mammal afflicted with said condition a pharmaceutically effective amount of a compound identified by the method of claim 1, 22, 23 or 24.

29. The therapeutic method of claim 28 wherein the specific cell type is lymphocyte, neutrophil, basophil, eosinophil or monotcyte.

30. The therapeutic method of claim 28 wherein the specific cell type is CD4 T cell lymphocyte, CD8 T cell lymphocyte or B lymphocyte.

31. The therapeutic method of claim 28 wherein the compound is etodolac.

32. A method of stimulating chemokinesis in a specific cell type of a mammal which comprises systemically administering a pharmaceutically effective amount of a compound identified by the method of claim 1, 22, 23 or 24.

33. The therapeutic method of claim 32 wherein the specific cell type is lymphocyte, neutrophil, basophil, eosinophil or monotcyte.

34. The therapeutic method of claim 32 wherein the specific cell type is CD4 T cell lymphocyte, CD8 T cell lymphocyte or B lymphocyte.

35. The therapeutic method of claim 32 wherein the compound is etodolac.

36. A method of treating inflammation in a specific cell type of a mammal which comprises stimulating chemokinesis in that cell type by systemically administering a pharmaceutically effective amount of a compound identified by the method of claim 1, 22, 23 or 24.

37. The therapeutic method of claim 36 wherein the specific cell type is lymphocyte, neutrophil, basophil, eosinophil or monotcyte.

38. The therapeutic method of claim 36 wherein the specific cell type is CD4 T cell lymphocyte, CD8 T cell lymphocyte or B lymphocyte.

39. A method of inhibiting malignant cancer cell metastatis in a mammal which comprises stimulating chemokinesis in an identified malignant cancer cell type by systemically administering a amount of a compound identified by the method of claim 1, 22, 23 or 24 which is effective to induce chemokinesis in said malignant cancer cell.

40. A method of depleting chronic lymphocytic leukemia cells in a mammal which comprises stimulating chemokinesis in chronic lymphocytic leukemia cells of a mammal by systemically administering to said mammal an amount of a compound identified by the method of claim 1, 22, 23 or 24 which is effective to induce chemokinesis in leukocytes associated with leukemia.

41. A method of inducing cytoskeletal changes in colon cancer cells of a mammal which comprises stimulating chemokinesis in a colon cancer cell type by systemically administering an amount of a compound identified by the method of claim 1, 22, 23 or 24 which is effective to induce chemokinesis in said colon cells.

42. A method of treating transplant rejection in a mammal which comprises stimulating chemokinesis in lymphocytes of said mammal by systemically administering to said mammal a pharmaceutically effective amount of a compound identified by the method of claim 1, 22, 23 or 24.

43. The therapeutic method of claim 42 wherein said lymphocyte is CD8 T cell lymphocyte.

44. The therapeutic method of claim 42 wherein the compound is etodolac.

45. A method of treating an allergy in a mammal which comprises stimulating chemokinesis in leukocytes of said mammal by systemically administering to said mammal a pharmaceutically effective amount of a compound identified by the method of claim 1, 22, 23 or 24.

46. The therapeutic method of claim 45 wherein said leukocyte is basophil or eosinophil.

47. The therapeutic method of claim 45 wherein said leukocyte is CD4 T cell lymphocyte.

48. The therapeutic method of claim 45 wherein the compound is etodolac.
